(19) United States
(12) Patent Application Publication
(10) Pub. No.: US 2025/0120843 A1
(43) Pub. Date: Apr. 17, 2025

(54) Footworn Device for Alleviating Pain Caused by Mortons Neuroma

(71) Applicant: SAM MATCHA, PINELLAS PARK, FL (US)

(72) Inventor: SAM MATCHA, PINELLAS PARK, FL (US)

(21) Appl. No.: 18/909,787

(22) Filed: Oct. 8, 2024

Related U.S. Application Data
(60) Provisional application No. 63/590,162, filed on Oct. 13, 2023.

(51) Int. Cl. A61F 5/14; A61F 5/01

(52) U.S. Cl. CPC A61F 5/14; A61F 5/0127

(57) ABSTRACT

A footworn device for alleviating pain caused by Morton's Neuroma comprising a sleeve, a metatarsal pad, and a toe cover. The metatarsal pad is coupled to a bottom-facing medial portion of the sleeve, approximately aligned with the arch of a user's foot. The toe cover is coupled to the end of the sleeve, encapsulating the user's toes. The toe cover is composed of a shrinkage resistant material as to not apply pressure to the toes, thereby pushing the toes together. Furthermore, the metatarsal pad is composed of a gel or fabric material to provide comfort and an upward pressure to the arch of a user's foot.

### FIELD OF THE INVENTION

The present invention relates generally to a footworn device. More specifically, the present invention is a footworn device for alleviating pain caused by Morton's Neuroma.

### BACKGROUND OF THE INVENTION

Morton's neuroma, also known as intermetatarsal neuroma and flat footedness, is a painful condition affecting the foot. It involves a thickening of tissue around a nerve leading to the toes, usually between the third and fourth metatarsal heads. The pressure and irritation on the nerve can cause severe discomfort, often described as a burning or sharp pain in the ball of the foot or between the toes. This condition is more common in women and can be exacerbated by wearing tight or high-heeled shoes, which squeeze the toes and increase pressure on the affected nerve.

Common symptoms of Morton's neuroma include numbness, tingling, or a feeling of having a pebble or small object inside the shoe. Pain typically worsens during activity or wearing certain types of footwear. Diagnosis often involves a physical examination and may include imaging studies like X-rays or MRI scans to confirm the presence and location of the neuroma.

Treatment options range from conservative measures like wearing wide, comfortable shoes, using orthotics, or administering corticosteroid injections to reduce inflammation and pain. In more severe cases, where conservative treatments don't provide relief, surgical removal of the neuroma may be considered. Early diagnosis and appropriate management can significantly alleviate symptoms and improve quality of life for individuals affected by Morton's neuroma.

The present invention addresses the need for effective pain relief and comfort for individuals suffering from Morton's neuroma. The present invention is a footworn device providing a targeted and adaptable solution to alleviate this pain and improve the daily lives of those affected.

The central design of this invention is a sleeve worn on the user's foot and leg, comprising a metatarsal pad, wherein said metatarsal pad is either removably attached or permanently fixed to the sleeve. The pad is designed to provide cushioning and support precisely to the metatarsals, reducing pressure and alleviating pain. This removable feature allows for customization and individualized comfort, catering to the unique needs and preferences of each user. Additionally, some embodiments of the invention may include a toe cover, providing added protection and support to the toes, enhancing the overall effectiveness of the device.

By combining the metatarsal pad and optional toe cover, the present invention aims to optimize relief for Morton's neuroma sufferers, promoting mobility, comfort, and an improved quality of life. The flexible and adaptable design ensures practicality and usability, making it a promising solution for managing the challenges posed by this prevalent foot condition.

One objective of the present invention is to alleviate pain caused by Morton's Neuroma. The present invention provides a removable metatarsal pad attached to a sleeve. The metatarsal pad of the present invention provides an upward force to the metatarsal of the user's foot to ensure proper spacing of the user's toes.

### SUMMARY OF THE INVENTION

The footworn device of the present invention comprises a sleeve and a metatarsal pad. In some embodiments of the present invention, the sleeve comprises a toe cover for protecting the toes of the user, whereby said toe cover is a loose, preshrunk, and thin material and thus does not push the toes of the user together.

### BRIEF DESCRIPTION OF THE DRAWINGS

FIG. 1 is a bottom view of one embodiment of the present invention on the foot of a user

FIG. 2. is a perspective view of one embodiment of the present invention on the foot of a user.

FIG. 3 is a table of the materials of the sleeve of the present invention.

FIG. 4 is a table of the materials of the metatarsal pad of the present invention.

FIG. 5 is a side view of one embodiment of the present invention.

FIG. 6 is a side view of an alternative embodiment of the present invention.

FIG. 7 is a perspective view of one embodiment of the present invention comprising a toe cover.

FIG. 8 is a table of the materials of the toe cover.

### DETAIL DESCRIPTIONS OF THE INVENTION

All illustrations of the drawings are for the purpose of describing selected versions of the present invention and are not intended to limit the scope of the present invention.

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

Other technical advantages may become readily apparent to one of ordinary skill in the art after review of the following figures and description. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Unless otherwise indicated, the drawings are intended to be read together with the specification, and are to be considered a portion of the entire written description of this invention. As used in the following description, the terms “horizontal”, “vertical”, “left”, “right”, “up”, “down” and the like, as well as adjectival and adverbial derivatives thereof (e.g., “horizontally”, “rightwardly”, “upwardly”, “radially”, etc.), simply refer to the orientation of the illustrated structure as the particular drawing figure faces the reader. Similarly, the terms “inwardly,” “outwardly” and “radially” generally refer to the orientation of a surface relative to its axis of elongation, or axis of rotation, as appropriate.

The present disclosure includes many aspects and features. Moreover, while many aspects and features relate to, and are described in the context of a footworn device, embodiments of the present disclosure are not limited to use only in this context.

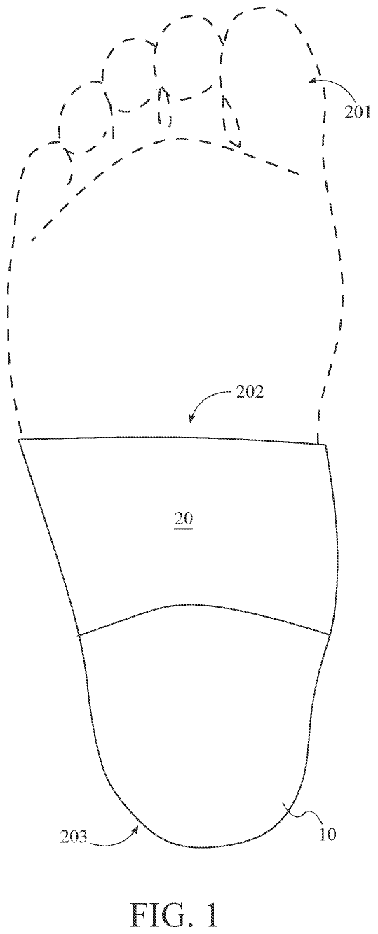

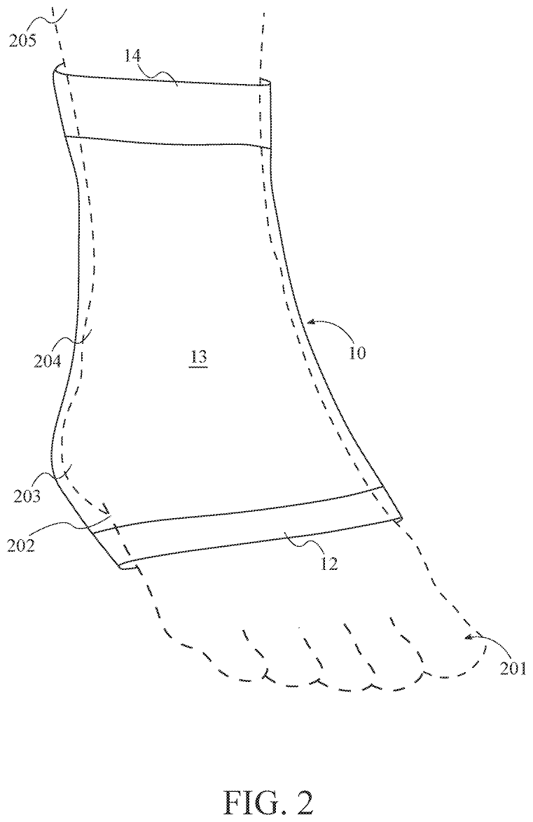

As shown in FIGS. 1-8, the present invention is a footworn device 1. In the context of the present invention, the footworn device 1 is secured to the foot of a user wherein, as shown in FIG. 1 and FIG. 2, wherein the foot of the user, as understood within the art, comprises a plurality of toes (i.e. toes) 201, an arch 202, a heel 203, an ankle 204, and a calf 205. In the preferred embodiment of the present invention, as shown in FIG. 1, the footworn device 1 comprises a sleeve 10 and a metatarsal pad 20, wherein the sleeve 10 covers a portion of the foot of the user, and the metatarsal pad 20 is coupled to the sleeve at a location approximately aligned within the arch 202 of the user.

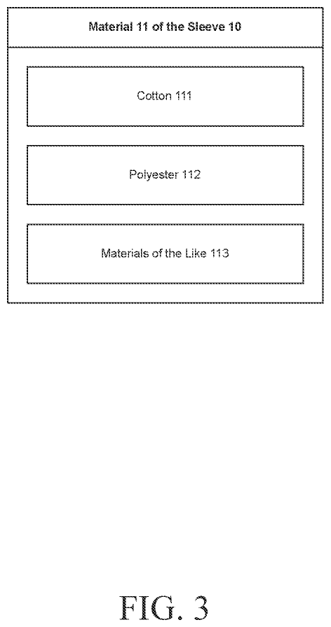

As shown in FIG. 2, in the preferred embodiment of the present invention, the sleeve 10 is a tubular member (also referred to herein as “a tubular body”) comprising a first end 12, a medial portion 13, and a second end 14. In the context of the present invention, the first end 12 and the second end 14 are opposing distal ends of the sleeve 10. Furthermore, within the context of the present invention, the first end 12 and the second end 14 are open ends, thereby forming a tube-shaped configuration of the sleeve 10. Moreover, in the preferred embodiment of the present invention, the medial portion 13 is the portion of the sleeve 10 that is interposed between the first end 12 and the second end 14. Although the sleeve 10 has been shown herein as covering a portion of the user's foot spanning from the calf 205 to the arch 202, in the context of the present invention, the sleeve 10 covers at least a portion of the user's foot, traversing from a location between the toes 201 and the arch 202 of the user, to a location between the arch 202 and the heel 203 of the user. As shown in FIG. 3, in the preferred embodiment of the present invention, the sleeve 10 is composed of a material 11 wherein said material is selected from cotton 111, polyester 112, and materials of the like 113.

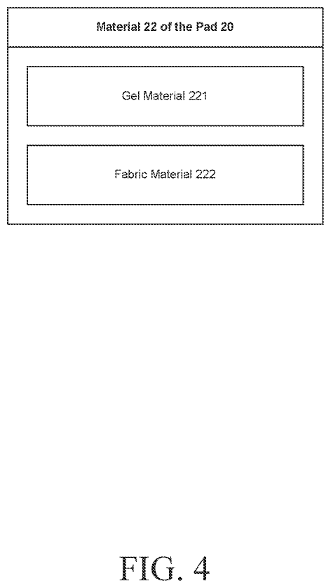

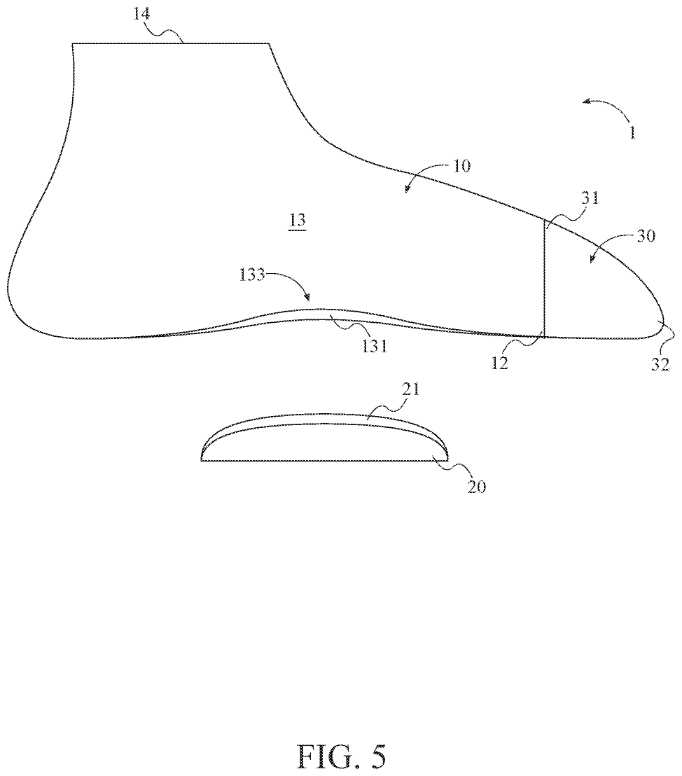

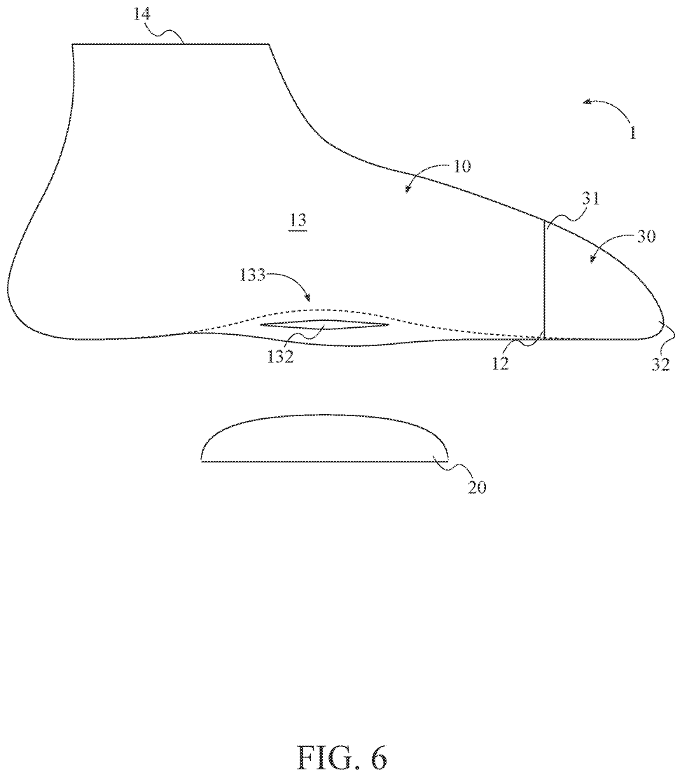

Within the preferred embodiment of the present invention, metatarsal pad 20 is further composed of a material 22, as shown in FIG. 4, wherein said material 22 is selected from: a gel material 221 and a fabric material 222. Moreover, as shown in FIG. 5 and FIG. 6, the metatarsal pad 20 is removably attachable to the sleeve 10. In the preferred embodiment of the present invention, the medial portion 13 of the sleeve 10 further comprises an arch 133, wherein said arch 133 is approximately aligned to the arch 205 of the user. Furthermore, in some embodiments, as shown in FIG. 5, the arch 133 of the medial portion 13 of the sleeve comprises a hook and loop fastener 131, and the metatarsal pad 20 comprises a hook and loop fastener 21, wherein the hook and loop fastener 131 of the medial portion 13 and the hook and loop fastener 21 of the metatarsal pad 20 are complimentary. In the context of the present invention, “complimentary” as used to describe the hook and loop fasteners 131,21, is intended to define a set of hook and loop fasteners that are attachable to each other. In some alternate embodiments of the present invention, the arch 133 of the medial portion 13 comprises a slot 132 wherein the metatarsal pad 20 is adjustably insertable into said slot 132.

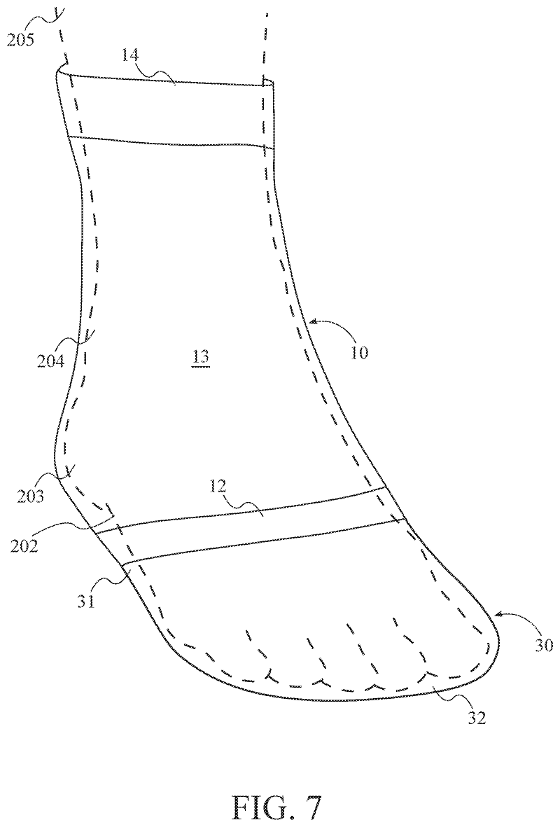

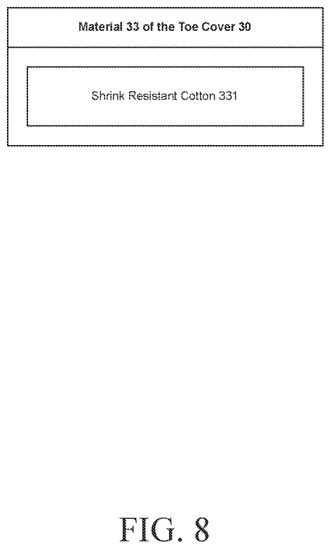

Furthermore, as shown in FIG. 5, FIG. 6, and FIG. 7, in some embodiments of the present invention, the footworn device 1 further comprises a toe cover 30. In the preferred embodiment of the present invention, the toe cover 30 comprises an open end 31 and a closed end 32, wherein said open end 31 and closed end 32 are opposed distal ends of the toe cover 30, whereby the open end 31 is coupled to the first end 12 of the sleeve 10 and the closed end 32 encapsulated the toes 201 of the user. In the preferred embodiment, as shown in FIG. 8, the toe cover 30 is composed a material 33 wherein said material 33 is resistant to deformations, specifically shrinkage, in the presence of varying moisture and temperature content. In such embodiments, the material 33 is a shrinkage resistant cotton 331.

Although the invention has been explained in relation to its preferred embodiment, it is to be understood that many other possible modifications and variations can be made without departing from the spirit and scope of the invention.

What is claimed is:

1. A footworn device comprising:
a sleeve;
a metatarsal pad; and
a toe cover;
wherein:
the sleeve is a tubular body comprising a first end;
the metatarsal pad is adjustably attached to a bottom portion of the sleeve; and
the toe cover is coupled to the sleeve at the first end of the sleeve.

2. The footworn device as claimed in claim 1, wherein the sleeve further comprises:
a second end; and
a medial portion;
wherein:
the first end and the second end of the sleeve are opposing distal ends of the sleeve;
each of the first end and the second end are open ends of the sleeve;
the medial portion is interposed between the first end and the second end; and
the metatarsal pad is coupled to the bottom portion of the medial portion of the sleeve.

3. The footworn device as claimed in claim 2, wherein the sleeve is composed of a material, whereby said material is selected from: cotton and polyester.

4. The footworn device as claimed in claim 1 wherein the pad is composed of a material, whereby said material is selected from: a gel material and a fabric material.

5. The footworn device as claimed in claim 2, wherein the toe cover comprises an open end and a closed end, wherein:
the open end is coupled to the first end of the sleeve; and
the closed end is an end opposite to the open end;
the closed end encapsulates the toes of a user.

6. The footworn device, as claimed in claim 5, wherein the toe cover is composed of shrink-resistant cotton.

7. The footworn device as claimed in claim 2 wherein:
the sleeve further comprises a hook and loop fastener;
the metatarsal pad further comprises a hook and loop fastener;
the hook and loop fastener of the sleeve is complimentary with the hook and loop fastener of the sleeve.

8. The footworn device as claimed in claim 2, wherein the medial portion of the sleeve comprises a slot wherein the metatarsal pad is insertable into said slot.

9. The footworn device as claimed in claim 7 wherein the medial portion of the sleeve further comprises an arch, wherein the hook and loop fastener of the sleeve is positioned on the arch.

10. The footworn device as claimed in claim 8 wherein the medial portion of the sleeve further comprises an arch, wherein the slot of the sleeve is positioned on the arch.

11. A footworn device comprising:
a sleeve;
a metatarsal pad; and
a toe cover;
wherein:
the sleeve comprises a hook and loop fastener;
the metatarsal pad comprises a hook and loop fastener;
the sleeve is a tubular body comprising a first end;
the metatarsal pad is adjustably attached to a bottom portion of the sleeve as the hook and loop fastener of the sleeve is complimentary to the hook and loop fastener of the metatarsal pad; and
the toe cover is coupled to the sleeve at the first end of the sleeve.

12. The footworn device as claimed in claim 11, wherein the sleeve further comprises:
a second end; and
a medial portion;
wherein:
the first end and the second end of the sleeve are opposing distal ends of the sleeve;
each of the first end and the second end are open ends of the sleeve;
the medial portion is interposed between the first end and the second end;
the medial portion comprises an arch wherein said arch of the medial portion is positioned at a foot arch of a user; and
the metatarsal pad is coupled to the bottom portion of the arch of the medial portion of the sleeve.

13. The footworn device as claimed in claim 12 wherein the toe cover comprises an open end and a closed end, wherein:
the open end is coupled to the first end of the sleeve; and
the closed end is an end opposite to the open end;
the closed end encapsulates the toes of the user.

14. The footworn device as claimed in claim 13 wherein the toe cover is composed of shrink-resistant cotton.

15. The footworn device as claimed in claim 13 wherein the sleeve is composed of a material, whereby said material is selected from: cotton and polyester.

16. The footworn device as claimed in claim 13 wherein the pad is composed of a material, whereby said material is selected from: a gel material and a fabric material.

17. A footworn device comprising:
a sleeve;
a metatarsal pad; and
a toe cover;
wherein:
the sleeve is a tubular body comprising a first end, a second end, and a medial portion;
the first end and the second end of the sleeve are opposing distal ends of the sleeve;
each of the first end and the second end are open ends of the sleeve;
the medial portion is interposed between the first end and the second end;
the toe cover comprises an open end and a closed end;
the open end is coupled to the first end of the sleeve; and
the closed end is an end opposite to the open end;
the closed end encapsulates the toes of the user; and
the metatarsal pad is removably coupled to the medial portion of the sleeve.

18. The footworn device as claimed in claim 17 wherein the medial portion of the sleeve comprises an arch wherein the arch of the medial portion is aligned with the arch of a user's foot;
the arch comprises a hook and loop fastener;
the metatarsal pad comprises a hook and loop fastener; and
the hook and loop fastener of the sleeve is complimentary with the hook and loop fastener of the arch.

19. The footworn device as claimed in claim 17 wherein the medial portion of the sleeve comprises an arch, whereby said arch of the medial portion is aligned with the arch of a user's foot, wherein:
the medial portion of the sleeve comprises a slot located at the arch of the medial portion; and
the metatarsal pad is adjustably insertable into said slot.

20. The footworn device as claimed in claim 19 wherein the metatarsal pad is composed of a gel material.
